(19) United States
(12) Patent Application Publication
(10) Pub. No.: US 2007/0077545 A1
(43) Pub. Date: Apr. 5, 2007

(54) Sperm protective polypeptides and uses thereof

(75) Inventors: Mathieu Boilard, Sainte-Julie (CA); Marc-Andre Sirard, Sainte-H elene-de-Breakeyville (CA); Pierre Leclerc, Sainte-Nicolas (CA); Janice Bailey, St-Augustin-de-Desmaures (CA); Catherine Lachance, Quebec (CA)

Correspondence Address: David S. Resnick;Nixon Peabody, 100 Summer Street, Boston, MA, 02110-2131, US

(73) Assignee: University Laval, Quebec (CA)

(21) Appl. No.: 10/575,698

(22) PCT Filed: Oct. 14, 2004
(86) PCT No.: PCT/CA04/01823

§ 371 Date: Sep. 26, 2006

Related U.S. Application Data
(60) Provisional application No. 60/510,549, filed on Oct. 14, 2003.

(51) Int. Cl. A01N 1/02; C07K 14/575

(52) U.S. Cl. 435/2; 530/350; 530/399

(57) ABSTRACT

The present invention relates to polypeptides capable of binding chaperone receptors, composition and methods for protecting, restoring, or improving the physiological properties of sperm cells. More particularly, the invention relates to protection of the motility, survival, fertility capability, resistance to cooling, freezing, and thawing of sperm cells put in contact with chaperone polypeptides.

### BACKGROUND OF THE INVENTION

a) Field of the Invention

The present invention relates to a group of proteins capable of preserving the physiological properties, such as viability, motility, fertility, or resistance to cooling, to freezing or thawing, of sperm cells, and most particularly mammalian sperm cells. Also, the invention relates to a composition comprising such a protein and a method of preserving the physiological properties of the sperm cells.

b) Description of the Prior Art

It has long been observed in nature that physiological properties of sperm cells, particularly mammalian sperm cells can be altered by different environmental factors.

The spermatozoon is a highly differentiated motile cell responsible for the meeting between the paternal and maternal genome moieties leading to the creation of a new descendant organism. In mammals, after ejaculation, the sperm cells travel through the female genital tract, and undergo a variety of physiological modifications. With these changes occurring, the sperm acquires the characteristics required for the fertilization. This phenomenon is known as capacitation. The uterus participates to the capacitation process through the action of uterine fluids and to the sperm transport by its contractions. Once the utero-tuval junction is crossed, the sperm are exposed to a new environment, that is to say the oviduct. Since breeding may occur several hours before ovulation, this little tube-shaped organ has a major task to accomplish in the fertilization process, which is to provide a sufficient number of live and competent spermatozoa to the ovum whenever the ovulate has occurred. It is known that fluids produced by this organ and specially proteins secreted by epithelial cells maintain sperm motility, viability and help to modulate the sperm capacitation. Also, it is known in several mammalian species that sperm cells bind to the apical plasma membrane from these oviduct epithelial cells (OEC) creating a reservoir of sperm cells. II rabbit, horse, and bovine, the results obtained by sperm co-incubation with apical membrane extracts from OEC indicated that the direct contact between epithelial and sperm cells is important for the maintenance of the motility and viability and to delay the capacitation. The biochemical link between the sperm and the OEC appears to be caused by the interaction between sperm lectins and oviduct epithelium fucose residues depending of species. These observations are however insufficient to understand the cellular mechanisms by which the oviduct ensures successful fertilization.

Although several factors in the semen itself or in the uterus or oviduct fluids were considered as being essentials in the success of carrying out fertilization in nature, this situation may significantly vary when the sperm cells are manipulated for assisted fertilization programs. For example, the sperm cells can be temporarily cooled down, or even frozen and thawed before performing the fertilization. Sperm injury is often manifested as loss of selective permeability, loss of integrity of the plasma membrane, outer acrosomal membrane and mitochondria. These manifestations are accompanied by loss of motility or viability, decreased energy production, changes to membrane lipid composition, loss of capability in binding and fusion to oocytes, and changes to membrane dynamic behavior.

In order to use sperm for artificial insemination, there is a need to prevent and repair loss of selective physiological features, such as permeability and loss of integrity of the plasma membrane, outer acrosomal membrane, and even may be mitochondria.

There is a need to develop factors, compositions and methods that may be used to prevent and even repair the damages caused to the physiological properties of sperm cells.

### SUMMARY OF THE INVENTION

One object of the present invention is to provide a polypeptide capable of binding a chaperon receptor for preserving or restoring a physiological property of sperm cells.

Another object of the invention is to provide composition comprising at least one polypeptide capable of binding chaperon receptor in an amount effective to preserve or restore at least one physiological property of sperm cells, and a physiologically acceptable carrier. The chaperone receptor binding polypeptide can be found in an amount is between 0.1 to 100 ng/ml of sperm diluent medium. Preferably, the concentration of chaperone receptor binding polypeptide is of between 1.0 to 25 ng/ml of sperm diluent medium. Once again, diluent medium as used herein can be the seminal liquid naturally produced by the reproductive system, or any other diluent generally known in the art to dilute sperm cells or the semen in preparation of fertilization, maturation, incubation freezing, thawing, or transfer into the female reproductive tract.

Those skilled in the art will recognize that the chaperone receptor binding polypeptide can also be used to preserve or restore physiological properties of sperm cells by applying it directly on, for example, a undiluted semen sample.

In accordance with another aspect of the present invention, there is provide a method for preserving or restoring a physiological property of sperm cells comprising contacting the sperm cells with a polypeptide capable of binding chaperon receptors.

The polypeptide or polypeptides that can be use to perform the different objects of the present invention may be comprised of at least one molecule selected from the group consisting of a chaperone polypeptide, a heat shock protein (HSP), a stress shock protein, a glucose regulated protein (GRP), a Sec A, a Sec B, a Sec Y, a GroEL, a matrix protein, a molecule capable of binding a sperm cell chaperone receptor, or analogs or fragments thereof.

Preferably the polypeptide can be, for example, HSP60, GRP 78, or a matrix protein that is a surface protein of epithelial cells, such as oviduct epithelial cells.

Depending on needs or situations, the method can be performed on fresh semen, frozen semen, or thawing or thawed semen.

Still another object of the present invention is the use of a polypeptide capable of binding a chaperon receptor in the preparation of a composition for preserving or restoring a physiological property of sperm cells.

The physiological property that can be preserved or restored according to the present invention is at least one of motility, movement characteristic, fertility, oocytes binding, fusion with an oocyte, viability, acrosome integrity, acrosome reaction, maturity, or resistance to at least one of cooling, freezing or thawing.

Preferably, the sperm cells for which physiological properties can be preserved and/or restore are mammalian sperm cells.

According to another object of the present invention there if provided the use of a polypeptide capable of binding a chaperon receptor in the preparation of a composition for preserving or restoring a physiological property of sperm cells.

### BRIEF DESCRIPTION OF THE DRAWINGS

FIG. 1 illustrates the identification of HSP60 and GRP78 as biotinylated proteins of OEC plasma membrane;

FIGS. 2A and 2B illustrate the immunoprecipitation of GRP78 and HSP60 from biotinylated (BIOT) or unbiotinylated (NB) OEC using anti-GRP78 (2A) and antiHSP60 (2B) antibodies;

FIGS. 3A to 3D illustrate the surface location of HSP60 on oviduct epithelial cells by indirect immunofluorescence;

FIG. 4 illustrates the co-immunoprecipitation of GRP78 and HSP60;

FIGS. 5A and 5B illustrate the effect of sperm-bound OAPM proteins on acrosomal loss (5A) and mortality (5B);

FIGS. 6A and 6B illustrate the effect of sperm-bound OAPM proteins on motility parameters;

FIG. 7 illustrates the autoradiography of one dimensional electrophoretic patterns of ³⁵S-radiolabelled proteins from oviduct epithelial cells;

FIGS. 8A to 8C illustrate the autoradiography of 2-D electrophoretic patterns of ³⁵S-radiolabelled proteins from oviduct epithelial cells;

FIGS. 9A to 9B illustrate the immunodetection of GRP78 and HSP60 in bull sperm incubated with fOAPM;

FIGS. 10A to 10C illustrate the assessment of frozen thawed sperm motility (10A), viability (10B) and acrosome integrity (10C) following incubation with a 10 ng/ml of HSP60;

FIG. 11 illustrates the assessment of frozen thawed sperm motility following 6 h incubation with a 10 ng/ml of GRP78; and

FIG. 12 illustrates the preservation of human sperm acrosome integrity during incubation with chaperone receptor binding polypeptides HSP60 and GRP78.

### DESCRIPTION OF THE PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

Through an extensive research work the present inventors have now found that sperm physiology can be preserved, restore, and even improved when contacted before or during manipulation with polypeptides binding to chaperone receptors at the surface of sperm cells.

Among molecular chaperones or chaperone receptor binding polypeptides that can be used to preserve and/or restore the physiological properties of sperm cells according to the present invention, such as for example but not limited to, viability, fertility, motility, acrosome integrity, oocytes binding and fusion, or maturation, mammalian or microbial chaperone proteins, analogs or fragments thereof selected from the group consisting of heat shock proteins (HSP 40, HSP 60, HSP 70, and HSP 90), stress shock proteins, matrix proteins, SecA, SecB, SecY, DnaK, DnaJ, TRiC, GroES, and GroEL can be considered.

It is known in the art that protein folding is governed solely by the protein itself, and that some proteins have helped in the process. This help consists of proteins called chaperones (or chaperonins) that are associated with the target protein during part of its folding process. However, once folding is complete (or even before) the chaperone will leave its current protein molecule and go on to support the folding of another.

Several lines of evidence suggest that chaperones' primary function may be to prevent aggregation. For example, a chaperone found in the ‘power plant’ organelles of mammalian cells (but otherwise similar to GroEL) has been shown to consist of 14 protein chains arranged as two doughnuts stacked on top of each other. The chaperoned protein sits inside the two doughnut holes, safely sequestered from other molecules with which it might aggregate.

The invention is also relating to pharmaceutical preparations or compositions comprising a sperm chaperone receptor binding polypeptide of proteinaceous nature which can be essentially pure, and activates, restore, or preserves sperm physiology together with any suitable excipients, diluent, medium or liquid carrier. Examples of suitable excipients are culture media or other physiological salt solutions.

The compositions or preparations are prepared according to methods known per se. The compositions or preparations according to the invention can be used in the treatment of infertility, as well in vivo as in-vitro.

The invention further comprises a method to preserve, restore, or improve the potential fertility, motility, sperm-egg binding, and other physiological properties necessary to complete a fertilization, of sperm by treating a sample with a chaperone protein according to the invention.

According to one embodiment of the present invention there is provided chaperone proteins, as sperm cell physiology preservative or improver, which can be found under a native or synthetic form, and which is useful for the following applications: a) as a pro-fertility additive to fluids used to suspend or re-suspend sperm at some point in the processing of semen for artificial insemination or for in vitro fertilization or similar assisted reproductive technology; b) as the active ingredient for a vaginal pro-fertility medication for self administration; c) as the basis for a reagent for use in quantifying the amount of chaperone proteins present on sperm to provide information related to the potential fertility of an individual spermatozoon or the population of sperm in a seminal sample; and d) as the antigen for the production of antibodies useful in predicting potential fertility.

In one embodiment, native or synthetic chaperone proteins are used to preserve, restore or improve the capacity of fresh or frozen-thawed sperm to fertilize eggs. Prior to artificial insemination, an aliquot of fresh or frozen-thawed sperm can be mixed with the chaperone proteins, which increases fertilizing capacity of the sperm. The molecular chaperones can alternatively be added directly into straws with sperm cells before freezing.

In another embodiment, native or synthetic chaperone protein or polypeptide can be used as a pro-fertility composition to be administered intra-vaginally as a semi-solid, liquid, or foam shortly before coitus. The composition might contain any chaperone protein or polypeptide, analogs or fragments thereof, as the active ingredient, suspended in an appropriate carrier which maintains biological activity and physiological properties, provides appropriate dispersion near the external cervical os, and facilitates effective and rapid partition of the active ingredient to the sperm after ejaculation.

The present invention will be more readily understood by referring to the following examples which are given to illustrate the invention rather than to limit its scope.

### EXAMPLE I

### Location of GRP78 and HSP70 on Oviduct Epithelial Cells

Materials and Methods

Oviduct Epithelial Cell Culture

Oviducts from cows in early estrous were collected at the slaughterhouse, maintained at 4° C. during transport and dissected from other tissues at the laboratory. Oviduct epithelial cells were recovered by stripping the oviducts and collecting the emerging fluid which contained the epithelial cells. These cells were washed by three successive sedimentations in Hanks medium (13.7 mM NaCl, 0.5 mM KCl, 450 μM NaHCO₃, 110 μM Na₂HPO₄, 40 μM KHPO₄, 5.5 mM D-Glucose, 5 mM PIPES, pH 7.4 with NaOH) containing 5% FBS (Medicorp, Montréal, Québec, Canada) and cultured at 38.5° C. and 5% CO₂ in TCM 199 (Earle's salts/Invitrogen™, Burlington, On, Canada) supplemented with 10% calf bovine serum (CBS); (ICN, Costa Mesa, Calif., USA), 0.2 mM pyruvate and 50 μg/mL gentamycin.

Apical Surface Localization of HSP60 and GRP78 by Affinity Precipitation

The apical surface localization of the identified proteins was confirmed using biotinylated “swimming” vesicles of cultured OEC from three different cows. Exclusive apical surface biotinylation was possible since cells are forming the swimming vesicles by exposing their ciliated apical surface. Apical surface biotinylation, precipitation and blot assay of the identified proteins were performed as previously described (Gorza and Vitadello, 2000, FASEB J., 14:461-475). Briefly, cultured OEC vesicles were rinsed three times with cold PBS, pH 8.0, and were next incubated on ice for 20 min in PBS in the presence of 1 mg Sulfo-NHS-LC-Biotin (Pierce, Rockford, Ill.) per ml. They were next rinsed three times with cold PBS. Total protein concentration in the sample was determined by BCA protein assay (Pierce) on an aliquot that had previously been precipitated by TCA and resolubilized. The biotinylated vesicles were homogenized in 500 μl RIPA buffer (0.15M NaCl, 1% Triton X-100, 0.5% sodium deoxycholate, 0.1% SDS, 50 mM Tris, 1 mM EDTA, pH 7.6) supplemented with protease inhibitors (17 μg/ml PMSF, 2 μg/ml leupeptin, 0.7 μg/ml pepstatin) for 30 min. The samples were centrifuged 20 min at 16 000×g to remove any cellular debris. Then, an enrichment of biotinylated proteins followed by immunoblotting using antibodies directed against specific proteins was performed.

Immobilized Neutravidin™ beads (Pierce) were added to the lysate and the mixture was incubated overnight at 4° C. The beads were washed four times in PBS+protease inhibitors. The beads were resuspended in 1-D loading buffer and heated at 100° C. for 10 min. Extracted proteins were subjected to 1-D SDS-PAGE and electrotransferred onto nitrocellulose membranes (Towbin et al., 1979, Proc. Natl. Acad. Sci. USA, 76:4350-4354). Non-specific binding sites were blocked by incubating the membranes in Tris buffered saline supplemented with Tween 20™ (TBST; 154 mM NaCl, 20 mM Tris pH 7.4, 0.1% Tween 20™) containing 5% (w/v) dry skimmed milk for 1 h. The presence of specific proteins was investigated by immunoblot using commercial antibodies directed against previously identified proteins. The membranes were washed three times with TBST. Then they were incubated for 1 h at room temperature the monoclonal antibodies directed against GRP78 or HSP60. Again, the membranes were washed and then incubated with a goat anti-mouse IgG conjugated to horseradish peroxidase for 45 min. At the end, the membranes were extensively washed by changing the TBST solution 6 times within a 30 min period. Immuno reactive bands were visualized by enhanced chemiluminescence using the ECL kit™ (Amersham Bioscience Corp. Baie d'Urfé, PQ, Canada) according to the manufacturer's instructions and film autoradiography.

Apical Surface Localization of the Proteins by Immunoprecipitation

An immunoprecipitation (IP) of specific proteins followed by affinity blot using horseradish peroxidase-conjugated avidin was achieved. In this experiment, a biotinylated cell lysate was created as described in the above section. This lysate was pre-cleared for 1 h using 1 μg of non-immune mouse IgG (Sigma) and 35 μl of protein G sepharose beads (Amersham Pharmacia Biotech), washed and resuspended in RIPA. The beads were next eliminated by centrifugation at 3000×g during 3 min. One microgram of either anti-HSP60 or anti-GRP78 was added and the samples were incubated for 2 h. Sepharose beads were then added and the samples were incubated for another 2 h. The samples were next centrifuged at 3000×g during 3 min, the beads were washed three times with RIPA buffer and were then heated at 100° C. in 1-D electrophoresis sample buffer for 5 min. The proteins were submitted to SDS-PAGE, transferred on nitrocellulose membrane and the biotinylation of HSP60 and GRP78 was assessed using horseradish peroxidase conjugated avidin and revealed by ECL and flim autoradiography.

Cell Surface Localization of HSP60 by Indirect Immunofluorescence

Oviduct epithelial cells from three different cows were cultured for two days after which the culture media were changed. The cells were then maintained in culture for three additional days. Half of the vesicle suspension was washed three times in a Hepes buffered Tyrode's medium (TLH), supplemented with 2 mg/mL BSA (TLHB). After that, they were incubated in the presence of 5 μg/mL of anti-HSP60 mouse monoclonal antibody or with a rabbit polyclonal antibody directed against isoforms 2 and 3 of Sarcoplasmic/Endoplasmic Reticulum Calcium ATPase (SERCA) provided by Dr. Jonathan Lytton (Department of Biochemistry & Molecular Biology University of Calgary, Alberta, Canada) for one hour. They were next washed trice in TLHB and incubated for 45 min with goat anti-mouse or with goat anti-rabbit IgG (respectively) conjugated to fluorescein-isothiocyanate (FITC). Finally, the vesicles were washed 6 times in TLHB, placed on a Poly-L-Lysine-coated coverslip and mounted on a glass slide. The other half of the vesicle suspension was washed three times in TLH containing 0.1% PVP40 (TLHP) and were fixed in PBS containing 3.7% formaldehyde for 10 min at room temperature. They were next rinsed three times in TLHP and permeabilized for 10 min in TLHP containing 0.2% Triton X-100™. Then, the cells were washed three times in TLHP and three times in TLHB. The indirect immunofluorescence against HSP60 or SERCA were performed as described for the non-permeabilized cells.

GRP78-HSP60 Interaction

Immunoprecipitation of HSP60 and GRP78 were performed on cell lysates, samples were submitted to SDS-PAGE and transferred using exactly the same protocol as described previously in this paper. The presence of GRP78 or HSP60 in each other IP sample was assessed by blotting the membrane of the HSP60 IP sample with the Anti-GRP78 monoclonal antibody and vice-versa using the protocol described above for western blotting.

Results

Apical Surface Localization of HSP60 and GRP78 by Affinity Precipitation

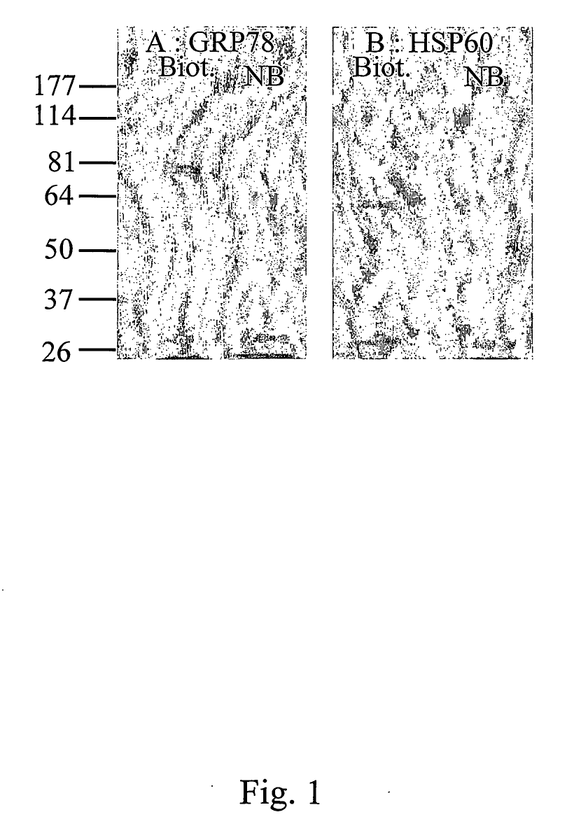

Because GRP78 is known to be principally localized in the endoplasmic reticulum and HSP60 is said to be mainly localized in mitochondria, both organelles unreachable by the sperm in vivo, experiments were conducted to determine whether or not these proteins were localized on the oviduct epithelial cell apical surface. The apical cell surface localization of HSP60 and GRP78 was confirmed by an affinity “pull-down” procedure followed by immunoblot using anti GRP78 or anti-HSP60 monoclonal antibodies. After cell surface biotinylation of the OEC vesicles which allows only the biotinylation of apical proteins, these two proteins were pulled down with neutravidin-conjugated beads suggesting that both GRP78 and HSP60 are expressed on the apical surface of oviduct epithelial cells (FIG. 1).

Apical Surface Localization of the Proteins by Immunoprecipitation

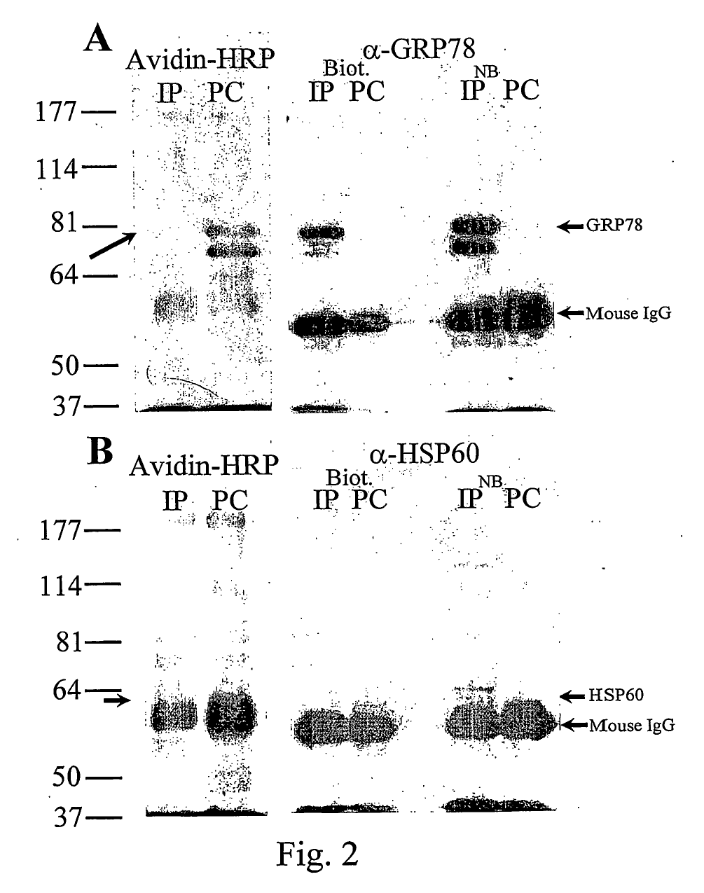

Conversely, to determine whether or not GRP78 and HSP60 are effectively biotinylated, protein extracts from cell-surface biotinylated OEC were immunoprecipitated using anti-GRP78 or anti-HSP60 monoclonal antibodies and their biotinylation status was assessed upon SDS-PAGE and transfer using peroxidase-conjugated avidin. Anti-GRP78 monoclonal antibody immunoprecipitated a biotinylated 78 kDa protein (FIG. 2A left side) which proved to be GRP78 as confirmed by western blot on the same membrane using the anti-GRP78 monoclonal antibody (FIG. 2A right side). Similarly, HSP60 also appeared to be biotinylated (FIG. 2B). In addition, previous results in our laboratory demonstrated that our commercial monoclonal antibody against HSP60 was less effective to detect its antigen in a western blot assay on apical plasma membrane proteins that were previously biotinylated (data not shown). As shown in the right panel of FIG. 2B, the biotinylation process interfered with the HSP60 recognition by this HSP60 antibody. Consequently, the small amount of HSP60 that was immunoprecipitated from biotinylated cells as compared to unbiotinylated cells strongly suggest that HSP60 was biotinylated.

Cell Surface Localization of HSP60 by Indirect Immunofluorescence

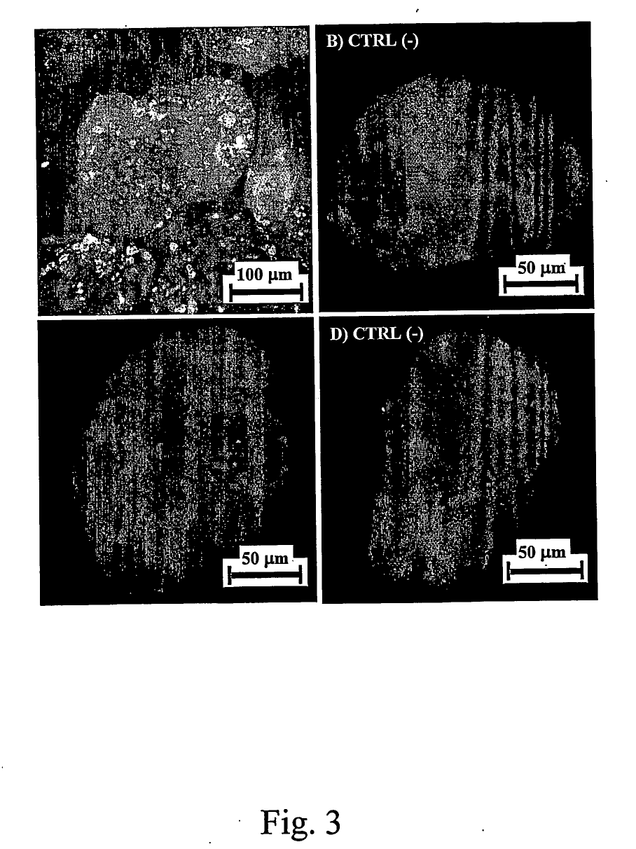

As the cell surface expression of HSP60 on OEC was not as clear as it was for GRP78 using IP, the hypothesis was also investigated using indirect immunofluorescence. HSP60, was detected on intact OEC (FIG. 3A) confirming again the cell surface location of this protein. The polyclonal antibody directed against a sarcoplasmic/endoplasmic reticulum calcium ATPase (SERCA)) failed to detect SERCA in non-permeabilized OEC (FIG. 3C), although a strong signal was observed when this antibody was used on permeabilized OEC.

GRP78-HSP60 Interaction

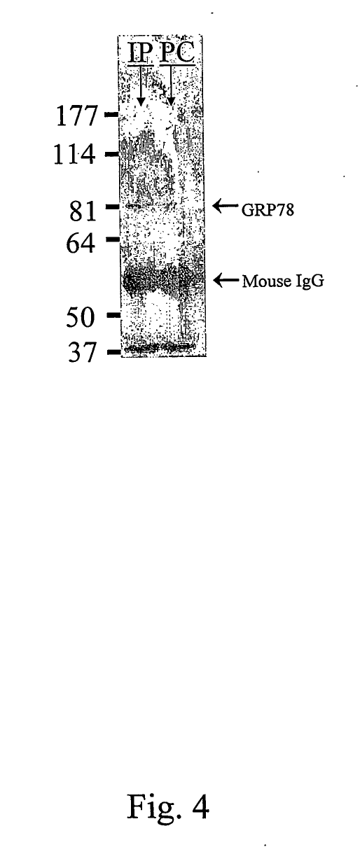

The possibility that HSP60 and GRP78 could associate together was assessed by verifying if HSP60 was able to co-immunoprecipitate GRP78 and, conversely, if the immunoprecipitation of GRP78 was pulling HSP60 down as well. The presence of HSP60 in the sample resulting from the IP of GRP78 was not detected (data not shown). On the opposite, a 80 kDa band is revealed by the anti-GRP78 antibody in the sample resulting from the immunoprecipitation of HSP60 (FIG. 4).

Conclusion

It was demonstrated that chaperone proteins are locates on the surface of healthy cells and allow the binding of the sperms.

### EXAMPLE II

### Improvement of Sperm Survival by Treatment with GRP78 and HSP60

Materials and Methods

Preparation of Apical Plasma Membranes

The preparation of fOAPM was done on the basis of what was previously described in Boilard et al. (2002, Biol. Reprod. 67:1125-1132). Briefly, oviducts from cows in early estrous were collected at the slaughterhouse, maintained at 4° C. during transport and dissected from other tissues at the laboratory. Oviduct epithelial cells were recovered by stripping the oviducts and collecting the emerging fluid which contained the epithelial cells. The cells were processed directly throughout the apical plasma membrane enrichment protocol (Behnke et al., 1990, Am. J. Physiol. 258:F311-320)] immediately after their recovery. In details, cells from eight oviducts were homogenized with a polytron aggregate homogenizer (Kinematica, Luzern, Switzerland) in 20 ml of buffer #1 (60 mM mannitol, 5 mM EGTA; all chemicals were from Sigma Chemical Company, St-Louis, Mo.), the pH was adjusted to 7.4 using a 1M Tris-HCl pH 7.4 solution). Then, 200 μl of 0.1M MgCl₂ was added to the homogenate, which was maintained on ice for 30 min to agglutinate the membranes of non-apical origin. A first centrifigation (3000×g) was performed at 4° C. for 15 min. The supernatant containing the apical membranes was removed and centrifuged at 27000×g for 30 min. The resulting supernatant was then removed and the pellet containing the membranes was re-suspended in 20 ml of buffer #2 (60 mM mannitol, 7 mM EGTA, pH 7.4 with Tris base) and homogenized with a Potter S homogenizer (Fisher Scientific). The mixture was then resubmitted to the purification steps involving incubation with MgCl₂ for 30 min and centrifugation at 3000×g and 27 000×g as described above. The pellet was re-suspended in 20 ml of buffer #3 (300 mM mannitol, pH 7.4 with 0.1M Tris-HCl pH 7.4) and again homogenized with the Potter S. The final mixture was pelleted for the last time at 27 000×g.

Apical plasma membranes from cultured OEC were also prepared. OEC were recovered as described above and washed by three successive sedimentations in Hanks medium (13.7 mM NaCl, 0.5 mM KCl, 450 μM NaHCO₃, 110 μM Na₂HPO₄, 40 μM KHPO₄, 5.5 mM D-Glucose, 5 mM PIPES, pH 7.4 with NaOH) containing 5% FBS (Medicorp, Montréal, Québec, Canada) and cultured at 38.5° C. and 5% CO₂ in TCM 199 (Earle's salts™/Invitrogen, Burlington, On, Canada) supplemented with 10% calf bovine serum (CBS); (ICN, Costa Mesa, Calif., USA), 0.2 mM pyruvate and 50 μg/mL gentamycin. After 16 h of incubation, OEC formed swimming vesicles with apical beating cils on the outer surface. The vesicles were separated from the culture media by a 50×g centrifugation for 2 min. cOAPM were obtained by running the apical plasma membrane enrichment protocol described above on these cultured cells except that the first 27 000×g pellet was homogenized directly into buffer # 3 and the apical material was pelleted again at 27 000×g instead of going through a second step of purification with buffer # 2. Protein concentration of each preparation was determined by the BCA protein assay (Pierce, Rockford, Ill., USA) and the amount of OAPM preparation used for any experiment as been quantified by the total amount of OAPM protein used.

Preparation of Radiolabelled OEC

OEC were cultured as previously described. All incubations required for labeling were done at 38.5° C. and 5% CO₂. The cells were resuspended in 70 ml of RPMI 1640 medium (ICN) and incubated for 15 min into a 75 cm² culture flask. They were then washed again by a 2 min centrifugation at 50×g. The final pellet was resuspended in RPMI 1640 containing 1% FBS (Medicorp) and 50 μCi/ml of radioactive amino acids (Tran³⁵S-Label™, ICN) and incubated for 3.5 h for radio labeling. The cells were then washed by centrifugation and a sample of radiolabeled OEC was dissolved into 50 μl of 1-D electrophoresis loading buffer (125 mM Tris-HCl pH 6.8, 4.6% SDS, 20% Glycerol, 87 μM Bromophenol blue, 10% β-mercaptoethanol) or with 250 μl of 2-D electrophoresis loading buffer (8 M Urea, 2% CHAPS, 0.5% IPG buffer for pH 3-10 linear isofocusing; Amersham Pharmacia Biotech, Piscataway, N.J.).

Sperm Preparation and Treatment

Frozen sperm samples were graciously provided by L'Alliance Semex Inc. (Guelph, ON, Canada) and the Centre d'Insémination Artificielle du Québec (Saint-Hyacinthe, PQ, Canada). For each experiment, straws containing pooled semen from 5 bulls were thawed in a water bath at 37° C. for 1 min. The semen was washed twice at 250×g for 10 min using a modified Tyrode medium supplemented with BSA (fraction V), pyruvic acid and gentamycin (Sp-TALP; Parrish et al., 1988, Biol. Reprod. 38:1171-1180) and sperm concentration was determined using a computer-assisted semen analyser (CASA) (Hamilton Thorne Research version 12.0f).

In the first set of experiment involving sperm, five millions motile washed sperm were added to each of the six 500 μl aliquots of Sp-TALP already containing a quantity of fOAPM equivalent to 150 μg of total fOAPM proteins. After 30 minutes of co-incubation at 38.5° C. and 5% CO₂, the entire volume was layered on top of a 45%/60% Percoll gradient (2 ml each) and was submitted to a 30 minutes centrifugation (700×g) to eliminate any material that was not strongly bound to the sperm cells. The pellet was washed by centrifugation in 5 ml of Sp-TALP at 370×g and layered on a second 45%/60% Percoll gradient, and centrifuged 30 minutes at 700×g. After the second Percoll wash, sperm were washed twice by centrifugation in Sp-TALP medium. Final sperm concentration was determined and adjusted to 25×10⁶ sperm/ml. Motility parameters were assessed using CASA after 0 h, 2 h, 6 h and 12 h of incubation at 38.5° C. and 5% CO₂. At the same time, the acrosomal integrity and viability were evaluated by eosin-nigrosin staining (Bamba, et al., 1988, Theriogenology, 29:1245-1251). Each motility, viability and acrosome reaction value was obtained by the analysis of a minimum of 100 sperm cells. This experiment was repeated three times on different days using different cOAPM preparations and different semen straws. Statistical analyses were done using the Statistical Analysis System (SAS Institute Inc., Cary, N.C., USA. Release 6.12) “proc glm” procedure for repeated times analysis.

In a second set of experiment with sperm, radioactive cOAPM were prepared from radiolabeled OEC as described above. Sperm were incubated with radio-labeled cOAPM as described earlier. The sperm pellet resulting from the second percoll gradient wash was washed again twice with cold phosphate buffered saline (PBS; 0.15 M NaCl, 10 mM NaH₂PO₄, pH 7.4) instead of Sp-TALP medium and the proteins were solubilized in either 1-D or 2-D electrophoresis loading buffer.

Protein Identification

The proteins were seperated either by 1-D SDS-PAGE [Laemmli, 1970 #52] or 2-D electrophoresis using 13 cm Immobiline DryStrip™ gels carrying an immobilized linear 3-10 pH gradient and the IPGphor Isoelectric Focusing System (Amersham Pharmacia Biotech) for the first dimension and SDS-PAGE for the 2^{nd} dimension. The gels were first fixed for 30 min in a 40% methanol and 10% acetic acid solution and than soaked for 30 min in Amplify (Amersham Pharmacia Biotech) to enhance radiation. The gels were next dried and subjected to autoradiography using Kodalc BioMax™ MR films. The mass and pI of labeled proteins bound to sperm were determined.

Proteins bound to sperm were localized on Coomassie brilliant blue stained 2-D gel of unlabelled freshly extracted OEC. This gel was duplicated and transferred on a PVDF membrane. The membrane was stained with Coomassie brilliant blue and proteins with mass and pI corresponding to ³⁵S-labelled proteins bound to spermatozoa were excised. Samples were brought to the Service Protéomique de l'Est du Québec (Centre Hospitalier Universitaire de Québec, pavillon CHUL, PQ, Canada) and were subjected to N-terminal sequencing by automatic Edman degradation performed on an Applied Biosystems model 473A pulsed liquid protein sequencer (Applied Biosystems, Foster city, Calif.).

Results

Modulation of Sperm Functions by Bound OAPM Proteins

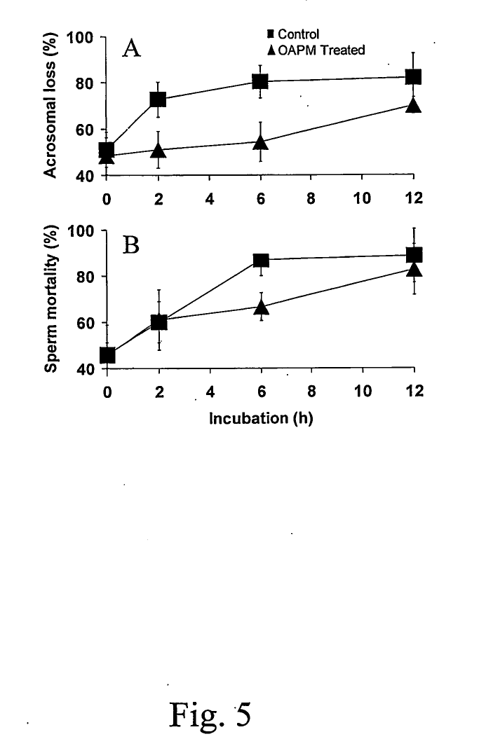

It is known that apical plasma membranes from the oviduct epithelium are the most efficient at modulating sperm function. The effect of the proteins from oviduct epithelial cells apical plasma membrane (OAPM) were first investigated. Conversely to previous experiments in which sperm have been analyzed following a 6 h incubation in the presence of OAPM, sperm were here pre-incubated in the presence of OAPM derived form cultured cells (cOAPM) or absence of cOAPM (control) and washed on two consecutive Percoll gradients to remove proteins that were not strongly bound. Then, these washed sperm carrying specific OAPM proteins were incubated. The objective was to verify the effect of bound proteins on different sperm parameters including viability, acrosomal integrity, and motility. After 2 h and 6 h of incubation, it clearly appeared that the cOAPM proteins that sperm carried had a protective effect on the acrosomal integrity since more acrosomal loss was observed in sperm pre-incubated in the absence vs. in the presence of cOAPM (73% and 80% vs. 51% and 54%, respectively FIG. 5A). However, this effect was transient since after 12 h of incubation, no significant difference was observed in the two sperm populations (cOAPM=70%, control=82%).

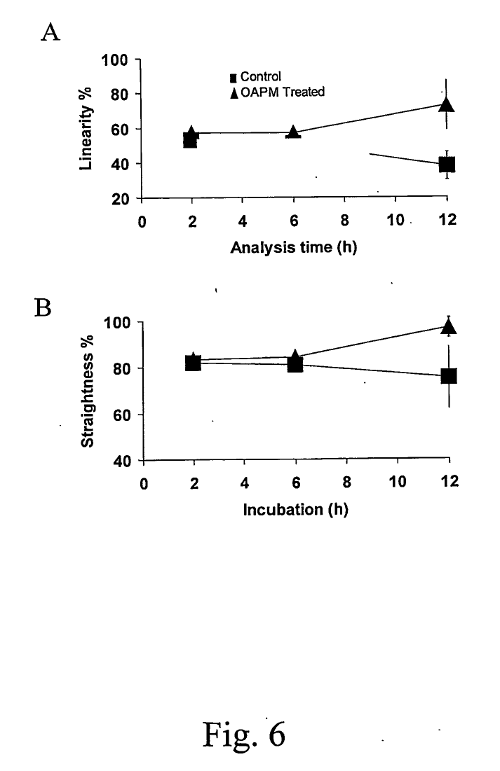

Moreover, bound cOAPM proteins also protected sperm viability. After a 6 h incubation, 87% of control sperm were dead as compared to 67% of those pre-incubated in the presence of cOAPM (FIG. 5B). However, no difference were noticed after 12 h of incubation. The effect of cOAPM proteins on sperm motility parameters were measured for up to 12 h with the first measurement done after 2 h. Of the many parameters analyzed, only the linearity and the straightness of the trajectory were influenced by the incubation with cOAPM (FIGS. 6A and 6B respectively). At 12 h, the linearity was higher in treated sperm (73%) than in untreated sperm (38%). After 12 h of incubation, the straightness was also higher in cOAPM-treated sperm (97%) than in untreated sperm (75%).

Identification of OAPM Sperm Binding Proteins

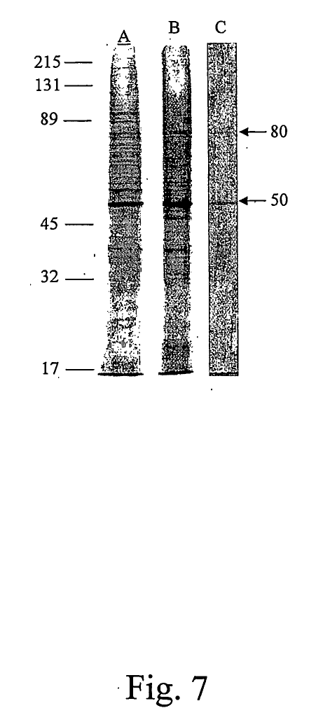

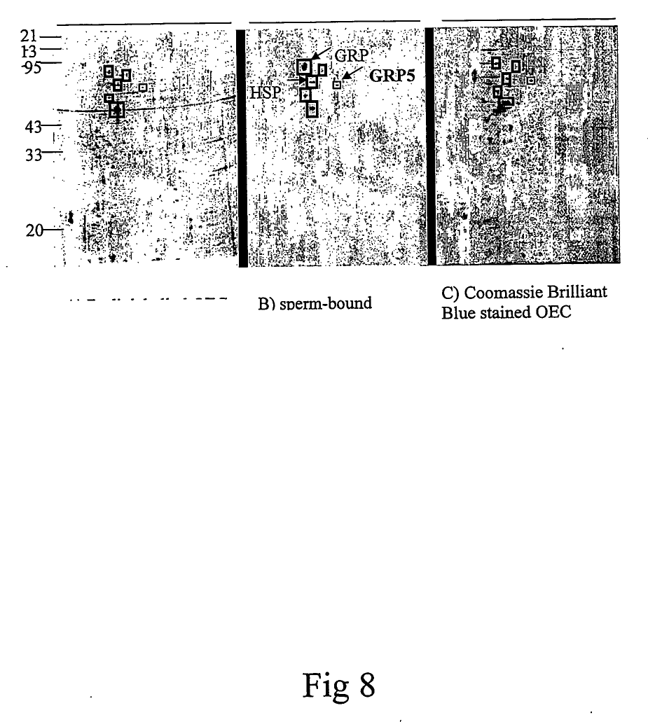

As shown by the protein pattern revealed by 1-dimensional (1-D) SDS-PAGE (FIG. 7), only few of the proteins labelled with ³⁵S in oviduct epithelial cells were present in the OAPM. It is also revealed that discontinued Percoll gradients are efficient to discard most of the unspecific binding of OAPM proteins to sperm because only two major bands of 50 and 80 kDa were observed (FIG. 7). The 2-dimensional (2-D) electrophoresis of radio-labelled OEC (FIG. 8A) and sperm incubated with radio-labelled cOAPM (FIG. 8B) revealed that 6 major proteins of approximate molecular weights and isoelectric point (MW/pI) of 80 kDa/5.0, 75 kDa/5.8, 60 kDa/5.5, 55 kDa/6.5, 50 kDa/5.0 and 45 kDa/5.1 strongly bound to sperm (FIG. 8B). Also, 5 additional proteins of 160 kDa/5.5, 60 kDa 6.2, 40 kDa/7.3, 37 kDa/7.3, 30 kDa/4.9 showed significant binding to the sperm cells.

The 6 major ³⁵S-labelled proteins (all included in boxes FIG. 8) were excised from the gel and submitted to the Edman degradation for N-terminal sequencing. Of these, 3 were subsequently identified (Table 1). The 80 kDa protein was identified as the glucose-regulated protein 78 (GRP78) also known as immunoglobulin heavy chain binding protein (BiP). Its identity was confirmed by western blots (not shown) using commercial monoclonal antibody (BD PharMingen, ON Canada). Similarly, the 60 kDa protein having a pI of 5.5 corresponded to the heat shock protein 60 (HSP60) and its identity was also confirmed by western blots (not shown) using a commercial monoclonal antibody (StressGen Biotechnologies Corp, BC, Canada). Finally, the 55 kDa/6.5 pI protein matched to some extent to the glucose-regulated protein 58 (GRP58) also known as protein disulfide isomerase (PDI) but none of numerous antibodies used against GRP58 (anti PDI donated by David Ferrari from Max Plank institute for biochemical research, Gottingen, Germany, Anti-PDI/SPA890 and Anti-ERP57/SPA-725 Stressgen Biotechnologies,) confirmed the identity of this protein.

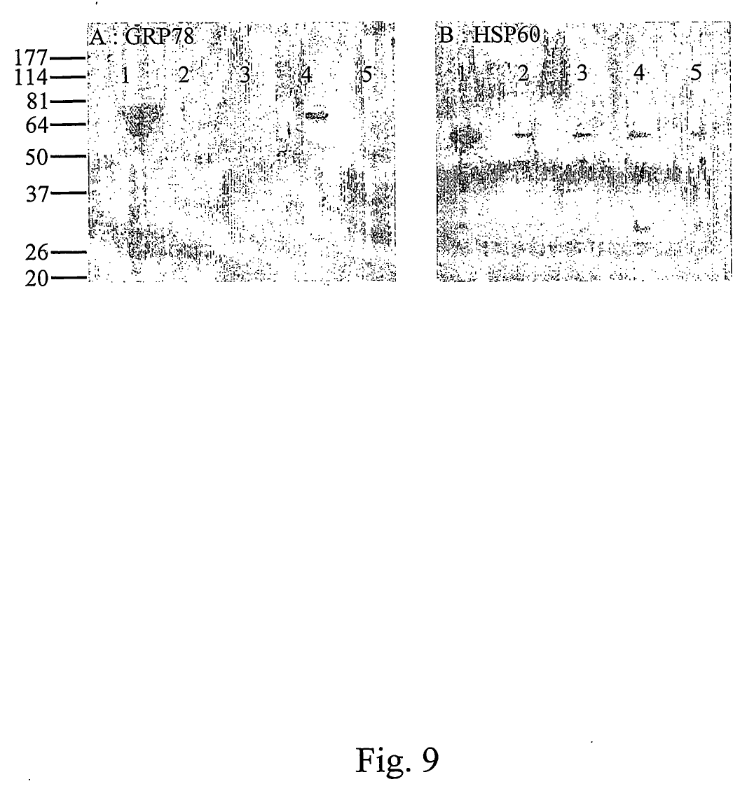

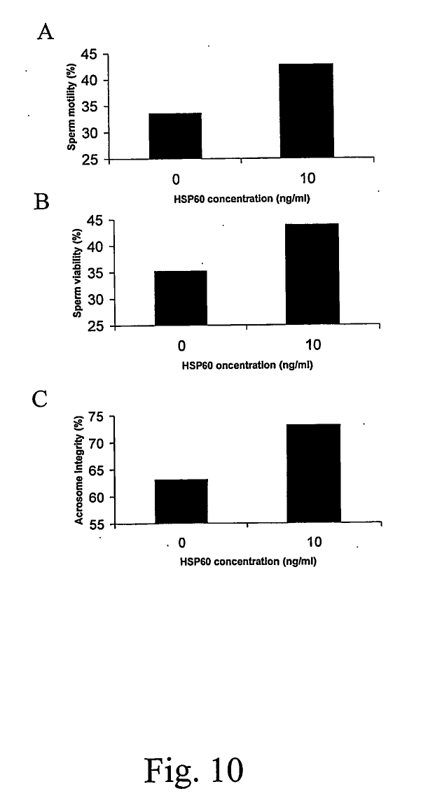

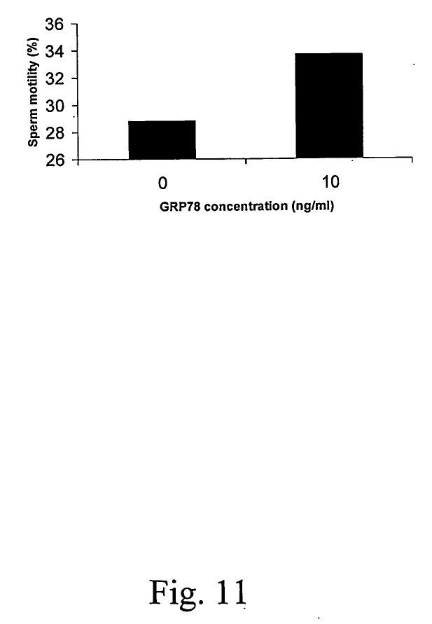

The binding of HSP60 and GRP78 to sperm was next confirmed using commercial antibodies. Sperm were co-incubated with OAPM derived from freshly extracted OEC (fOAPM) and washed on Percoll gradients. Their proteins were separated by electrophoresis and the presence of HSP60 and GRP78 was detected by western blotting. As shown in FIG. 9A, GRP78 is absent from freshly ejaculated or cryopreserved bull sperm. However, GRP78 is abundantly present on sperm previously incubated with fOAPM, which confrrms that GRP78 was acquired by sperm upon co-incubation with apical extracts from OECs. With a similar procedure, it can be shown that although HSP60 is present in sperm (FIG. 9B), a higher level of HSP60 was detected when sperm were incubated with fOAPM confiing that, as for GRP78, HSP60 is acquired by sperm during incubation with OAPM. In addition, the cryopreservation process did not affect the presence of this heat shock protein in bull sperm (FIG. 9B).

Conclusion

In the present experiment, sperm-bound OAPM proteins, by themselves, had a positive effect on the viability, acrosomal integrity as well as the linearity and straightness of sperm movement and that it allows to modulate sperm motility and intracellular calcium concentration ([Ca⁺⁺]ᵢ).

The results presented in this experiment demonstrate a novel cellular mechanism for the protection and preservation of physiological properties of sperm via a solid interaction or protein transfer from the OAPM to the sperm cells.

### EXAMPLE III

### Assessment of Human Sperm Cells

Materials and Methods

Human and bovine sperm proteins were solubilized in SDS-PAGE sample buffer, separated by gel electrophoresis, transferred to nitrocellulose, then incubated with biotinylated commercial recombinant GRP78 and HSP60 proteins. After extensive washes, the membrane was incubated with streptavidin conjugated to horseradish peroxidase, and the sperm proteins that bind to GRP78 or HSP60 were detected by enhances chemiluminescence and autoradiographic film exposure.

Assessment of the level spontaneously acrosome reaction of human sperm cells occurring during incubation. Data were obtained as performed for bovine sperm cells described above.

Results

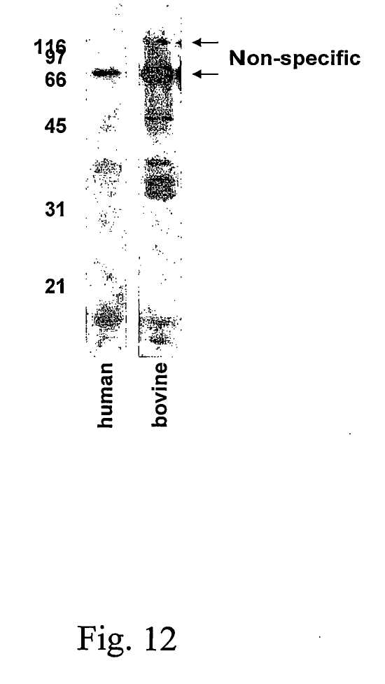

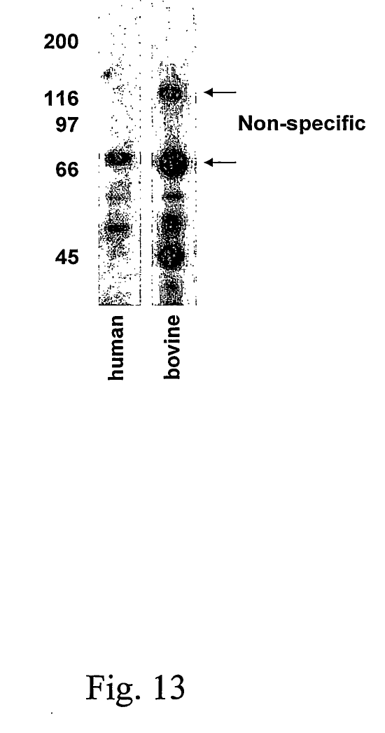

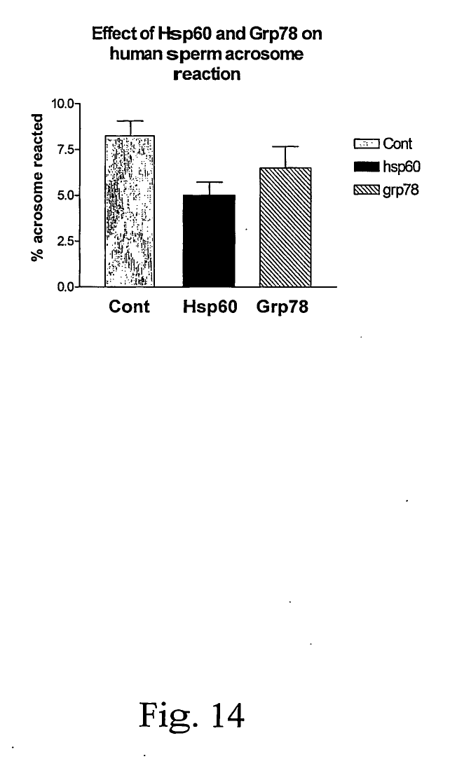

The non-specific bands represent proteins that bind to streptavidin even in the absence of biotinylated GRP78 (FIG. 12).

After extensive washes, the membrane was incubated with streptavidin conjugated to horseradish peroxidase, and the sperm proteins that bind to HSP60 were detected by enhances chemiluminescence and autoradiographic film exposure. The non-specific bands represent proteins that bind to streptavidin even in the absence of biotinylated HSP60 (FIG. 13).

FIG. 14 shows that human sperm cells integrity is better preserved from acrosome reaction when placed in a culture medium containing HSP60 or GRP78 proteins when compared to sperm cells incubated in a plain medium.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within lcnown or customary practice within the art to which the invention pertains and as may be applied to the essential features hereinbefore set forth, and as follows in the scope of the appended claims.

We claim:

1. A polypeptide capable of binding a chaperon receptor for preserving, restoring or improving a physiological property of sperm cells.

2. The polypeptide of claim 1, comprising at least one molecule selected from the group consisting of a chaperone polypeptide, a heat shock protein (HSP), a stress shock protein, a glucose regulated protein (GRP), a Sec A, a Sec B, a Sec Y, a GroEL, a matrix protein, a molecule capable of binding sperm cell chaperone receptor, analogs thereof and fragments thereof.

3. The polypeptide of claim 2, wherein said HSP is HSP60.

4. The polypeptide of claim 2, wherein said GRP is GRP 78.

5. The polypeptide of claim 2, wherein said matrix protein is a surface protein of epithelial cells.

6. The polypeptide of claim 5, wherein said epithelial cells are oviduct epithelial cells.

7. The polypeptide of claim 1, wherein said physiological property is at least one of motility, movement characteristic, fertility, oocytes binding, fusion with an oocyte, viability, acrosome integrity, acrosome reaction, maturity, and resistance to at least one of cooling, freezing and thawing.

8. The polypeptide of claim 1, wherein said sperm cells are mammalian sperm cells.

9. The polypeptide of claim 1, being in concentration of between 0.1 to 100 ng/ml of sperm diluent medium.

10. The polypeptide of claim 1, being in concentration of between 1.0 to 25 ng/ml of sperm diluent medium.

11. A composition comprising at least one polypeptide capable of binding chaperon receptor in an amount effective to preserve, restore or improve at least one physiological property of sperm cells, and a physiologically acceptable carrier.

12. The composition of claim 11, wherein said amount is between 0.1 to 100 ng/ml of sperm diluent medium.

13. A method for preserving, restoring or improving a physiological property of sperm cells comprising contacting said sperm cells with a polypeptide capable of binding chaperon receptors.

14. The method of claim 13, wherein said polypeptide comprises at least one molecule selected from the group consisting of a chaperone polypeptide, a heat shock protein (HSP), a stress shock protein, a glucose regulated protein (GRP), a Sec A, a Sec B, a Sec Y, a GroEL, a matrix protein, a molecule capable of binding a sperm cell chaperone receptor, analogs, and fragments thereof.

15. The method of claim 13, wherein said contacting is performed on fresh semen, frozen semen or thawed semen.

16. (canceled)
